(19) United States
(12) Patent Application Publication
(10) Pub. No.: US 2025/0185942 A1
(43) Pub. Date: Jun. 12, 2025

(54) Nasal/Oral Sampling Cannula

(71) Applicant: Ngah Chai LEOW, PETALING JAYA (MY)

(72) Inventor: Ngah Chai LEOW, PETALING JAYA (MY)

(21) Appl. No.: 18/798,773

(22) Filed: Aug. 8, 2024

(30) Foreign Application Priority Data
Dec. 6, 2023 (MY) PI2023007438

(51) Int. Cl. A61B 5/097; A61B 5/00

(52) U.S. Cl. CPC A61B 5/097; A61B 5/6819; A61B 5/682

(57) ABSTRACT

A nasal cannula has a tube configured with a first part defining an inhalation pathway and a second part defining an exhalation pathway. The first part and the second part are divided by a sealing member. The first part has an inlet and a first nasal prong for insufflating gas into the nostril of a patient. The second part has an outlet, a second nasal prong for collecting exhaled gas from the nostril of a patient, and a first means for releasing pressure build-up along the exhalation pathway.

### CROSS-REFERENCE TO RELATED APPLICATIONS

This application claims priority to, and the benefit of, Malaysian Patent Application No. PI2023007438, filed on Dec. 6, 2023, the contents of which are incorporated by reference herein in their entirety.

### TECHNICAL FIELD

The present invention relates to generally to medical devices, and more particularly to a nasal cannula that mitigates occlusion of nasal prongs during oxygen gas insufflation and carbon dioxide gas sampling.

### BACKGROUND ART

When a human patient is sick or undergoes surgery, it is often necessary to supplement the body's inhalation with a treatment gas, such as oxygen gas or a gaseous anaesthetic. In these instances, an accurate quantitative determination of the amount of at least one gaseous component, such as carbon dioxide, in the blood flowing through the pulmonary alveoli of the human patient is high desirable. In intensive care conditions or under general anaesthetic, an accurate measurement of the composition of the breathing gas in the pulmonary alveoli allows bodily functions of a patient to be more readily supervised and treatment of the patient more favourably adapted to the state of those functions. Accurate measurements of at least one gaseous component in the exhalation of a human patient can help improve related diagnostic methods for determining bodily conditions.

One area of particular interest is the monitoring of end-tidal carbon dioxide, which is the partial pressure of the carbon dioxide component of exhaled gas at the end of exhalation in a spontaneously breathing patient. The quantitative monitoring of end-tidal carbon dioxide in spontaneously breathing patients who are unintubated (those not requiring intubation with an endotracheal tube) would be particularly useful for those unintubated patients who whilst awake are being treated with supplemental oxygen administration and are receiving regional or local anaesthesia or are in a recovery room during emergence from residual general anaesthesia.

Commonly, a nasal cannula is used to provide a flow of gases to a patient via the patient's nostrils or other nasal passages if required. Such device is configured to either deliver oxygen in to or draw carbon dioxide out from the nostrils of the patient for end-tidal carbon dioxide measurement. Nasal cannula assemblies generally consist of entry tubing, either symmetric or single sided that lies across the upper lip. Protruding from this tubing are a pair of open-ended prongs which extend into the nostrils of the patient to deliver oxygen. Nasal cannulas are advantageous in the sense that they are more comfortable and acceptable to be used than a face mask to most patients. The technologies related to nasal cannula assemblies are generally established and are disclosed in the prior art.

One example of a nasal cannula configured to deliver a flow of fluid to a patient is disclosed U.S. Pat. No. 5,335,656A. Disclosed herein is a nasal cannula which has a wall member cooperating with a hollow body of the cannula to define inhalation and exhalation manifolds and sealingly engaging the hollow body to provide a gas-tight seal for positively preventing fluid communication between these manifolds, and a hollow nasal prong protruding from each manifold to be received in a corresponding nostril of a patient. Another example is disclosed in surface of a patient's nose to secure the patient interface. Another example is disclosed in U.S. Pat. No. 11,420,002B2 describing a nasal cannula comprising a manifold part having an inlet to receive the flow of fluid and at least one outlet to deliver the flow of fluid to the patient's nostrils, a port located on the manifold part to deliver a medicament into the flow of fluid being delivered by the nasal cannula to the patient. U.S. Pat. No. 7,353,826B2 also describes a ventilation interface comprising a nasal cannula body having a pair of nasal prongs location on a top portion of the nasal cannula body and a bellows-like structure configured to contact a bottom surface of the nose to create a sealing interface between the nasal cannula body and the nose.

A normal nasal cannula is designed with the nasal prongs having a slightly smaller outer diameter as it is anatomically desirable to ensure comfort when inserted into the nostrils of the patient. This is also important for imparting proper direction and flow rate of delivering gas into the nasal cavities of the patient. Nevertheless, problems can be encountered if the patient is in the horizontal or prone position which tends to cause accumulation of secretions in the nasal cavities. It can be a particularly dire problem if the secretions dry up and thereby clog the openings of the nasal prongs. This ultimately occludes the oxygen gas insufflation or carbon dioxide sampling. For example, use of the nasal cannula for monitoring end-tidal carbon dioxide during the administration of anaesthesia. As such, there exists a need to provide a nasal cannula which mitigates the incidence of occlusion of the nasal prongs. Such nasal cannula may be useful for sampling of carbon dioxide and insufflation of oxygen gas. The present invention eliminates the abovementioned drawbacks and provides a solution to the drawbacks.

### BRIEF SUMMARY

One aspect of the invention is to provide a nasal cannula for sampling carbon dioxide gas exhaled by a patient. Advantageously, the nasal cannula of the present invention mitigates the incidence of occlusion of the tip of the nasal prong during withdrawal of carbon dioxide gas by the nasal cannula to a carbon dioxide monitoring device. This minimizes the risk of distorting end-tidal carbon dioxide measurements.

Another aspect of the invention is to provide a nasal cannula for insufflating a patient with oxygen gas whilst accurately monitoring end-tidal carbon dioxide. Ideally, the nasal cannula will resume to function properly for its intended purpose when either or both nasal prongs become occluded due to accumulation of nasal secretions.

At least one of the preceding objects is met, in whole or in part, in which the embodiment of the present invention describes a nasal cannula comprising a tube configured with a first part defining an inhalation pathway and a second part defining an exhalation pathway, the first part and the second part are divided by a sealing member, the first part comprising an inlet and a first nasal prong for insufflating gas into the nostril of a patient, and the second part comprising an outlet, a second nasal prong for collecting exhaled gas from the nostril of a patient, and a first means for releasing pressure build-up along the exhalation pathway.

In a preferred embodiment of the present invention, it is disclosed that the sealing member is a partitioning wall between the first part and the second part for preventing gas commingle between the inhalation pathway and the exhalation pathway.

In a preferred embodiment of the present invention, it is disclosed that the tube further comprises a means for collecting a portion of orally exhaled gas from the mouth of a patient.

Preferably, the tube has a channel having an opening and in fluid communication with the exhalation pathway for delivering the portion of orally exhaled gas to the second part of the tube.

In a preferred embodiment of the present invention, the channel further comprises an extended lid part configured to intercept the orally exhaled gas from the mouth of a patient and pass thereof to the opening of the channel.

Preferably, the partitioning wall is located proximate to the channel and adjacent to the first nasal prong.

In a preferred embodiment of the present invention, the first means for releasing pressure build-up is at least an aperture disposed on the exhalation pathway adjacent to the partitioning wall and aligns with the first and second nasal prongs.

In a preferred embodiment of the present invention, the first and second nasal prongs, each is configured with a second means for releasing pressure build-up therealong.

Preferably, the second means for releasing pressure build-up comprises one or more apertures disposed at the centre on a top surface and a bottom surface of the first and second nasal prongs.

Preferably, the inlet of the first part is configured to connect with an external oxygen source.

Preferably, the outlet of the second part is configured to connect with a vacuum pump.

More preferably, the vacuum pump is further coupled to a device for measuring concentration or partial pressure of gas exhaled by a patient.

One skilled in the art will readily appreciate that the present invention is well adapted to carry out the objects and obtain the ends and advantages mentioned, as well as those inherent therein. The embodiment described herein is not intended as limitations on the scope of the invention.

### BRIEF DESCRIPTION OF THE DRAWINGS

For the purpose of facilitating an understanding of the invention, there is illustrated in the accompanying drawing the preferred embodiments from an inspection of which when considered in connection with the following description, the invention, its construction and operation and many of its advantages would be readily understood and appreciated.

FIG. 1 shows a top perspective view of a nasal cannula according to the present invention.

FIG. 2 shows a bottom perspective view of a nasal cannula according to the present invention.

FIG. 3 shows a side perspective view of a nasal cannula of FIG. 1 according to the present invention.

FIG. 4 shows a side perspective view of a nasal cannula of FIG. 2 according to the present invention.

FIG. 5 shows a back view of a nasal cannula according to the present invention.

FIG. 6 shows a front view of a nasal cannula according to the present invention.

FIG. 7 shows a cross sectional view of a nasal cannula according to the present invention, taken along the line A-A of FIG. 1.

FIG. 8 depicts the inhalation pathway and exhalation pathway in the nasal cannula according to the present invention.

### DETAILED DESCRIPTION

Hereinafter, the invention shall be described according to the preferred embodiments of the present invention and by referring to the accompanying description and drawings. However, it is to be understood that limiting the description to the preferred embodiments of the invention is merely to facilitate discussion of the present invention and it is envisioned that those skilled in the art may devise various modifications without departing from the scope of the appended claim.

The present invention relates to a nasal cannula that is to be used in connection with a source of oxygen gas at one end and in connection with a vacuum pump coupled to a device for monitoring carbon dioxide gas at another end. The nasal cannula of the present invention is suitable for insufflating a patient with oxygen gas whilst accurately monitoring end-tidal carbon dioxide.

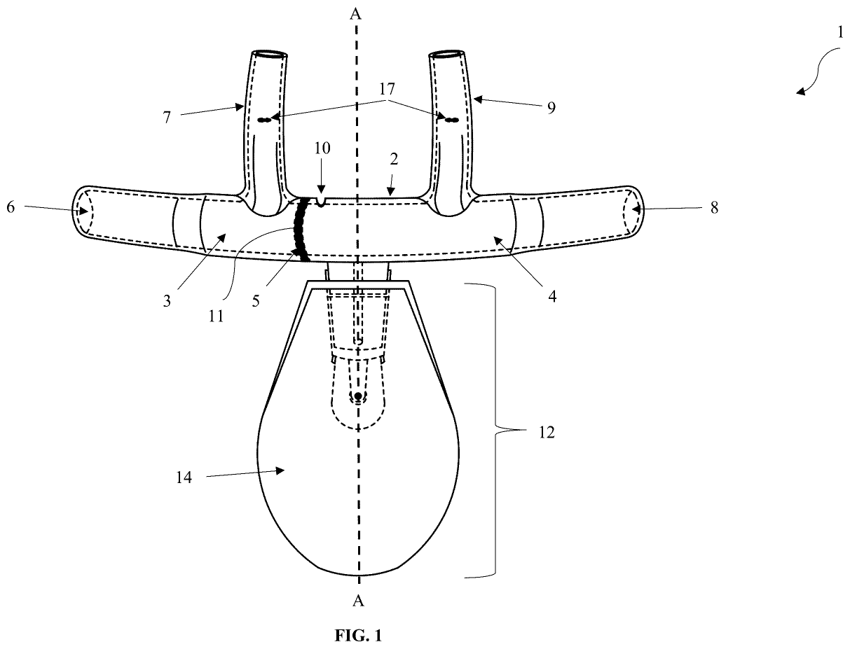

FIG. 1 shows a preferred embodiment of the nasal cannula, generally designated by the reference numeral (1), having a tube (2) configured with a first part (3) defining an inhalation pathway and a second part (4) defining an exhalation pathway. In the context of the present invention, the term “inhalation pathway” refers to a route in the nasal cannula (1) through which a medical gas, such as oxygen gas is delivered from oxygen concentrator, oxygen cylinder or any other oxygen supply equipment and directed to a patient's respiratory system. On the other hand, the term “exhalation pathway” refers to a route in the nasal cannula (1) through which carbon dioxide gas is expelled from a patient's respiratory system and directed to a device for monitoring carbon dioxide levels. In the preferred embodiment, the first part (3) of the tube (2) defining the inhalation pathway comprises a first nasal prong (7) for insufflating oxygen gas into the nostril of a patient. In the preferred embodiment, the second part (4) of the tube (2) defining the exhalation pathway comprises a second nasal prong (9) for collecting exhaled gas such as carbon dioxide gas from the nostrils of a patient. It is to be understood that the first and second nasal prongs (7, 9) are adapted to fit within a corresponding nostrils of a patient.

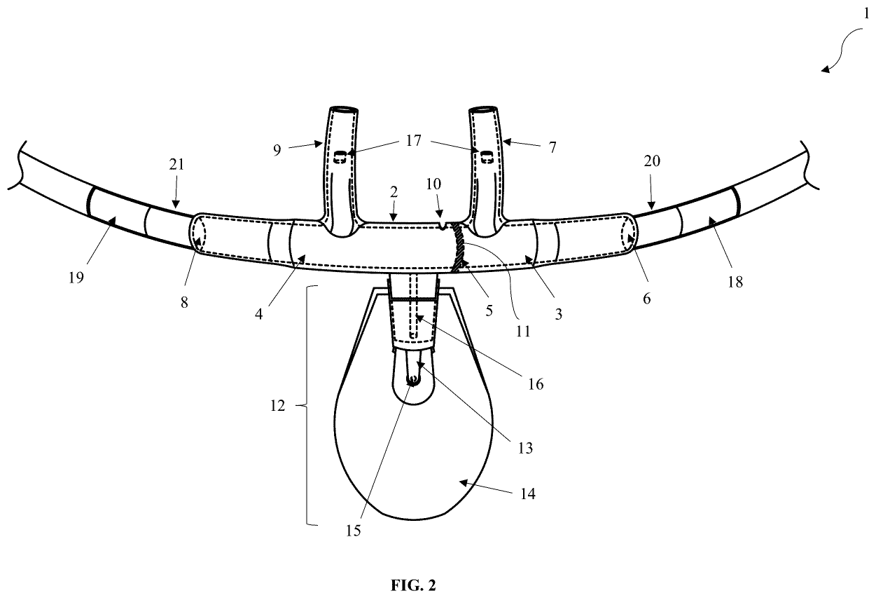

Sealingly engaging the inner surface of the interior of the substantially hollow tube (2) is a sealing member (5) which serves as an intermediate transverse barrier dividing the tube (2) into the first part (3) and the second part (4). The sealing member (5) may be made of a material that is substantially impermeable to liquids and gases, thereby preventing fluid communication between the inhalation pathway and the exhalation pathway. The entire nasal cannula (1) of the present invention is preferably moulded from a flexible plastic material whereby the sealing member (5) is integrally moulded with the wall of the tube (2) as seen best in FIGS. 1 and 2. Nevertheless, the sealing member (5) may be a separate barrier sealingly adhered to the wall of the tube (2) by other means, such as by an adhesive composition or by fusion of the material of the sealing member (5) to the material of the tube (2) by solvent welding, sonic welding or the like. In a preferred embodiment of the present invention, the sealing member (5) is a partitioning wall (11) between the first and second parts (3, 4) of the tube (2), as can be seen in FIGS. 1 and 2. In the preferred embodiment, the partitioning wall (11) is provided to prevent commingling of oxygen gas and carbon dioxide gas between the inhalation pathway and exhalation pathway.

Referring to FIG. 2, the first part (3) of the tube (2) defining the inhalation pathway may be connected to an external source of oxygen gas (not shown) such as oxygen concentrator, oxygen cylinder or any other oxygen supply equipment by a first supply tube (18) via an inlet (6) which extends and terminates at an end of the first part (3). On the other hand, the second part (4) of the tube (2) defining the exhalation pathway may be connected to a vacuum pump of a device for measuring concentration or partial pressure of carbon dioxide (not shown) by a second supply tube (19) via an outlet (8) which extends and terminates at an end of the second part (4). The first and second supply tube (18, 19) are preferably flexible and may be obtained separately or may be provided as part of the nasal cannula (1), in which case, an end portion (20) of the first supply tube (18) and an end portion (21) of the second supply tube (19) may be permanently fixed by adhesion to the corresponding inlet (6) and outlet (8) of the tube (2). For example, the supply tube ends (20, 21) may be sealingly fixed in place by means similar to those for sealingly securing the sealing member (5) within the tube (2). On the other hand, if the nasal cannula (1) of the present invention is to be supplied without attached supply tubes, the tube (2) is preferably made of a material that is more elastic than the supply tube ends (20, 21) such that the force required for seating the supply tube ends (20, 21) will provide air-tight sealing engagement between the respective supply tube ends (20, 21) and the inlet (6) and outlet (8).

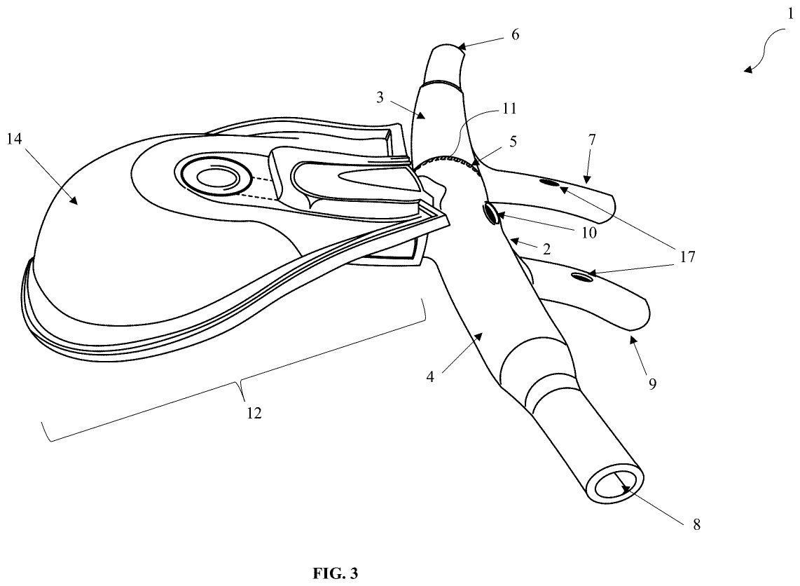

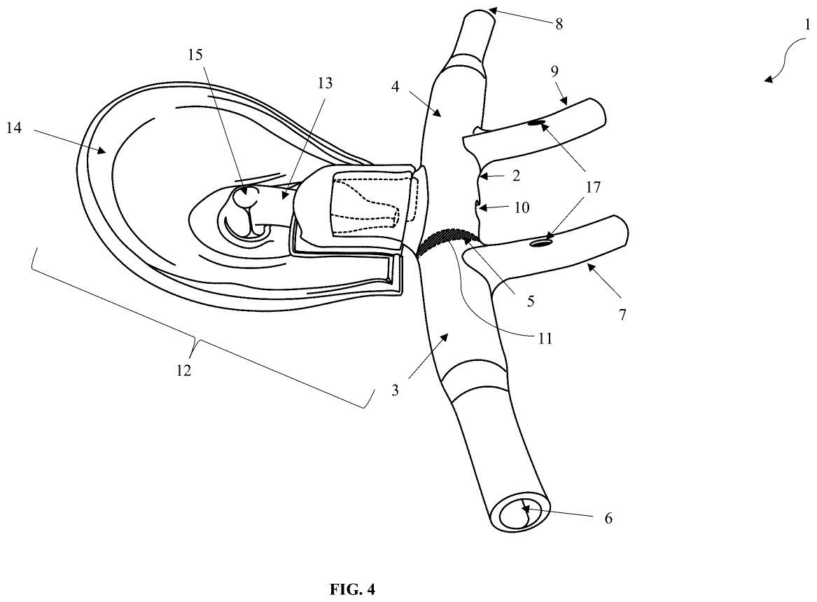

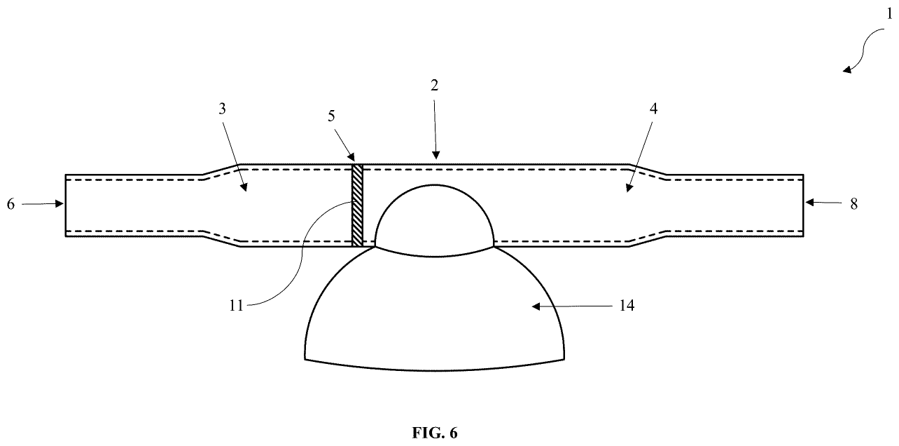

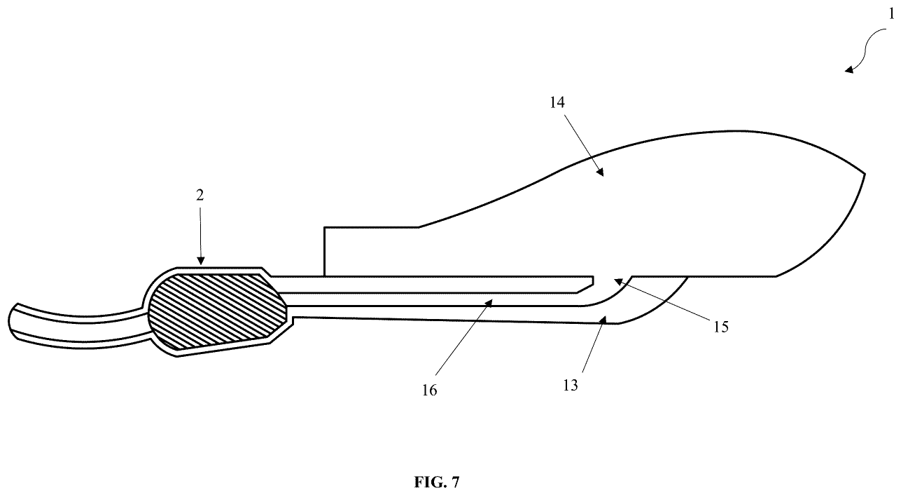

To ensure maximum sampling of gas exhaled from the respiratory system of a patient, it is ideal to collect, to a certain extent, a portion of gas exhaled orally from the mouth of a patient for carbon dioxide monitoring and measurement. Referring to FIGS. 1-4, the tube (2) of the nasal cannula (1) of the present invention comprises a means for collecting a portion of orally exhaled gas (12) from the mouth of a patient. In the context of the present invention, the orally exhaled gas includes carbon dioxide. The means for collecting a portion of orally exhaled gas (12) comprises a channel (13) having an opening (15) and an extended lid part (14) integrally disposed on the channel (13). As best seen in FIGS. 4 and 6, the channel (13) extends in a transverse direction of the tube (2) and in fluid communication with the exhalation pathway of the second part (4) for delivering the portion of orally exhaled gas from the mouth of a patient to the second part (4) of the tube (2). Referring to FIGS. 1 and 2, proximate to the channel (13) is the partitioning wall (11) that separates the first and second parts (3, 4) of the tube (2). The partitioning wall (11) is also located adjacent to the first nasal prong (7). Such positioning of the partitioning wall (11) allows optimum space for disposition of the channel (13) in fluid communication with the exhalation pathway of the second part (4). In a preferred embodiment of the present invention, the channel (13) further comprises an extended lid part (14) as abovementioned that is provided for positioning adjacent the mouth of a patient to intercept orally exhaled gas and pass at least a portion of the orally exhaled gas to the opening (15) of the channel (13). Interception of orally exhaled gas from the mouth of a patient is made possible due to the inwardly curved shape of extended lid part (14), as shown in FIG. 4. The channel (13) can also been seen in FIG. 1 illustrated with dotted lines, indicating that the view of the channel (13) is obstructed by the extended lid part (14), which is disposed thereon. As illustrated in FIG. 7, the opening (15) of the channel (13) allows the orally exhaled gas from the mouth of a patient to be received and delivered into the second part (4) of the tube (2) via an air passageway (16) located within the channel (13).

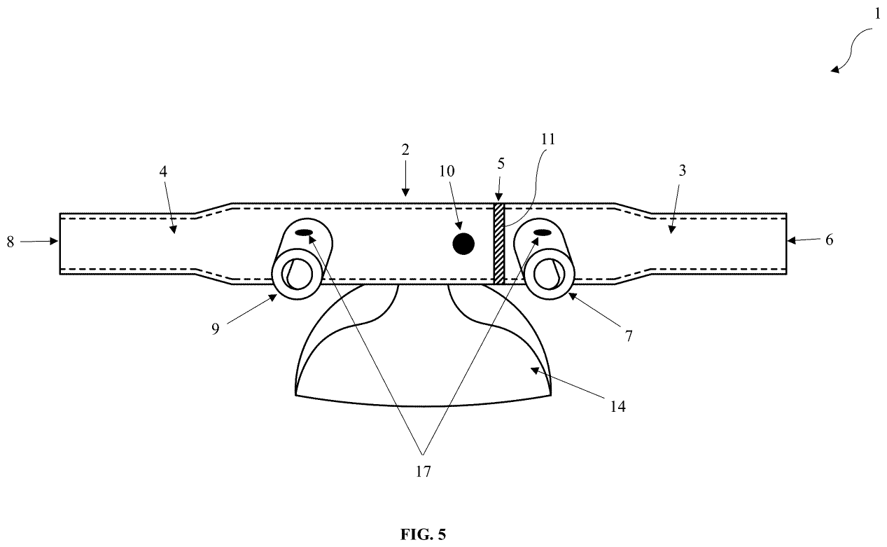

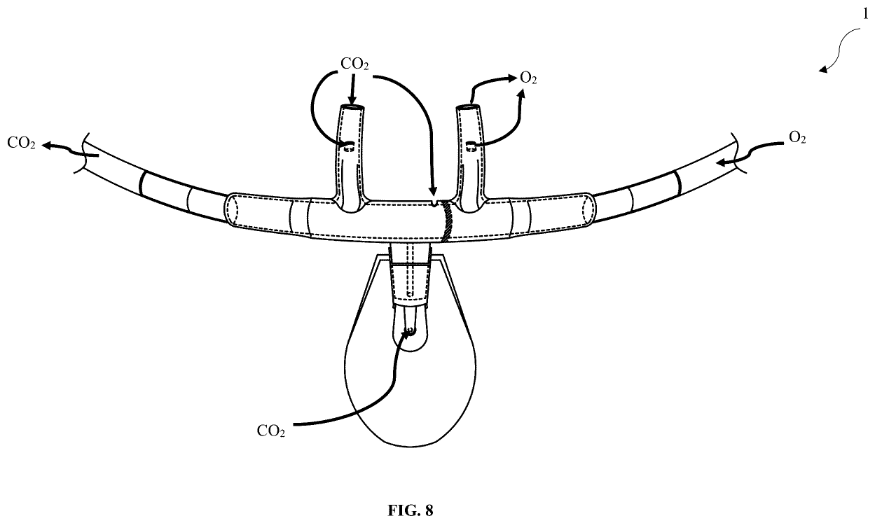

During usage of a conventional nasal cannula, insertion of nasal prongs into the nostrils should not typically cause irritation and discomfort to a patient. However, some patients may be more sensitive than others, and prolonged insertion of nasal prongs into the nostrils of a patient can potentially result in irritation of the nasal cavities, for example when the patient is in the horizontal or prone position. Nasal irritation causes secretion of nasal fluids in the nostrils, in which accumulation thereof could inevitably clog the nasal prongs. Occlusion of the nasal prongs could increase pressure in the nasal cannula and prevents the nasal cannula from functioning properly. In order to mitigate or eliminate the incidence of occlusion of the nasal prongs, the second part (4) of the tube (2) of the nasal cannula (1) comprises a first means for releasing pressure build-up (10) along the exhalation pathway. As can be seen generally in FIGS. 1-4, the first means for releasing pressure build-up (10) is at least an aperture disposed on the exhalation pathway of the second part (4) of the tube (2). The working mechanism of the first means for releasing pressure build-up (10) is best illustrated in FIG. 8. In the incidence whereby the second nasal prong (9) is occluded, pressure build-up from the carbon dioxide gas in the exhalation pathway of the second part (4) of the tube (2) can be relieved via the aperture of the first means for releasing pressure build-up (10). When in use with a vacuum pump connected to the outlet (8) of the second part (4), pressure build-up along the exhalation pathway can be relieved through the aperture (10) provided. If desired, one or more apertures (10) may be provided along the exhalation pathway. Furthermore, carbon dioxide gas exhaled from the nostrils of a patient may be received by the aperture (10) when the outlet (8) of the second part (4) is connected to a vacuum pump, as shown in FIG. 8. Referring to FIG. 5, the aperture (10) is situated along the second part (4) of the tube (2). Preferably, the aperture (10) is situated opposite to the channel (16) or in vicinity with the partitioning wall (11). Preferably, the aperture (10) aligns parallel to the first and second nasal prongs (7, 9).

In the present invention, a second means for releasing pressure build-up (17) is provided to mitigate the incidence of occlusion of the first and second nasal prongs (7, 9). In a preferred embodiment of the present invention, the first and second nasal prongs (7, 9), each is configured with another aperture (17) for releasing pressure build-up therealong, as shown in FIGS. 1 and 2. Each of the apertures (17) is disposed at the centre on a top surface and a bottom surface of the first and second nasal prongs (7, 9). Similarly, carbon dioxide gas exhaled from the nostrils of a patient may be received by the aperture (17). On the other hand, in the incidence whereby the first nasal prong (7) is occluded, the aperture of the second means for releasing pressure build-up (17) allows delivery of oxygen gas therefrom into the nostril of a patient, as best seen in FIG. 8.

The present disclosure includes as contained in the appended claims, as well as that of the foregoing description. Although this invention has been described in its preferred form with a degree of particularly, it is understood that the present disclosure of the preferred form has been made only by way of example and that numerous changes in the details of construction and the combination and arrangements of parts may be resorted to without departing from the scope of the invention.

What is claimed is:

1. A nasal cannula comprising:
a tube configured with a first part defining an inhalation pathway and a second part defining an exhalation pathway, the first part and the second part are divided by a sealing member;
the first part comprising an inlet and a first nasal prong for insufflating gas into the nostril of a patient; and
the second part comprising an outlet, a second nasal prong for collecting exhaled gas from the nostril of a patient, and a first means for releasing pressure build-up along the exhalation pathway.

2. The nasal cannula according to claim 1, wherein the sealing member is a partitioning wall between the first part and the second part for preventing gas commingle between the inhalation pathway and the exhalation pathway.

3. The nasal cannula according to claim 1, wherein the tube further comprises a means for collecting a portion of orally exhaled gas from the mouth of a patient.

4. The nasal cannula according to claim 3, wherein the tube has a channel having an opening and in fluid communication with the exhalation pathway for delivering the portion of orally exhaled gas to the second part of the tube.

5. The nasal cannula according to claim 4, wherein the channel further comprises an extended lid part configured to intercept the orally exhaled gas from the mouth of a patient and pass thereof to the opening of the channel.

6. The nasal cannula according to claim 1, wherein the partitioning wall is located proximate to the channel and adjacent to the first nasal prong.

7. The nasal cannula according to claim 1, wherein the first means for releasing pressure build-up is at least an aperture disposed on the exhalation pathway adjacent to the partitioning wall and aligned with the first and second nasal prongs.

8. The nasal cannula according to claim 1, wherein the first and second nasal prongs are each configured with a second means for releasing pressure build-up therealong.

9. The nasal cannula according to claim 8, wherein the second means for releasing pressure build-up comprises one or more apertures disposed at the centre on a top surface and a bottom surface of the first and second nasal prongs.

10. The nasal cannula according to claim 1, wherein the inlet of the first part is configured to connect with an external oxygen source.

11. The nasal cannula according to claim 1, wherein the outlet of the second part is configured to connect with a vacuum pump.

12. The nasal cannula according to claim 11, wherein the vacuum pump is further coupled to a device for measuring concentration or partial pressure of gas exhaled by a patient.
